(19) United States
(12) Patent Application Publication
(10) Pub. No.: US 2023/0285110 A1
(43) Pub. Date: Sep. 14, 2023

(54) MARKER INSERTER

(71) Applicant: Merit Medical Systems, Inc., South Jordan, UT (US)

(72) Inventors: Vince Tangherlini, South Jordan, UT (US); Emilio Aguilar, South Jordan, UT (US); Steven Wolf, South Jordan, UT (US)

(21) Appl. No.: 18/182,792

(22) Filed: Mar. 13, 2023

Related U.S. Application Data
(60) Provisional application No. 63/269,311, filed on Mar. 14, 2022.

(51) Int. Cl. A61B 90/00; A61B 17/34

(52) U.S. Cl. CPC A61B 90/39; A61B 17/3468; A61B 2090/3987

(57) ABSTRACT

A lockable device, and methods for using said device, for inserting a marker into a patient's body. The device may be inserted into a patient's body in the locked state, transitioned into an unlocked state, and then used to deliver a marker to a precise location. The device may provide tactile and audible feedback once the marker has been placed and indicate that the device is ready for removal.

This application claims priority to U.S. Provisional Application No. 63/269,311, filed on Mar. 14, 2022 and titled, “MARKER INSERTER,” which is hereby incorporated by reference in its entirety.

### TECHNICAL FIELD

### Brief Description of the Drawings

The written disclosure herein describes illustrative embodiments that are non-limiting and non-exhaustive. Reference is made to certain of such illustrative embodiments that are depicted in the figures, in which:

FIG. 1 is an exemplary embodiment of a marker insertion device.

FIG. 2 is an exploded view of an exemplary embodiment of the marker insertion device of FIG. 1.

FIG. 3 is an exemplary embodiment of a first handle member of the marker insertion device of FIG. 1.

FIG. 4 is an exemplary embodiment of an elongate member of the marker insertion device of FIG. 1.

FIG. 5 is an exemplary embodiment of a cannula of the marker insertion device of FIG. 1.

FIG. 6 is an exemplary embodiment of a second handle member of the marker insertion device of FIG. 1.

FIG. 7 is an exemplary embodiment of a stylet of the marker insertion device of FIG. 1.

FIG. 8 is an exemplary embodiment of a first assembly of the marker insertion device of FIG. 1.

FIG. 9 is an exemplary embodiment of a second assembly of the marker insertion device of FIG. 1.

FIG. 10 is a cross-sectional view of an exemplary embodiment of the first and second assemblies when coupled.

FIG. 11 is an exemplary embodiment of the device of FIG. 1 in a first position.

FIG. 12 is an exemplary embodiment of the device of FIG. 1 in a translating position.

FIG. 13 is an exemplary embodiment of the device of FIG. 1 in a second position.

FIG. 14 is an exemplary embodiment of a notch of the elongate member of the insertion device of FIG. 1.

FIG. 15 is an exemplary embodiment of a raised portion of the elongate member of the insertion device of FIG. 1.

FIG. 16A is cross-sectional view of an exemplary embodiment of the distal tip of the device of FIG. 1 in a first position.

FIG. 16B is cross-sectional view of an exemplary embodiment of the distal tip of the device of FIG. 1 in a second position.

FIG. 17 is a front perspective view of the marker insertion device of FIG. 1.

FIG. 18 is a rear perspective view of the marker insertion device of FIG. 1.

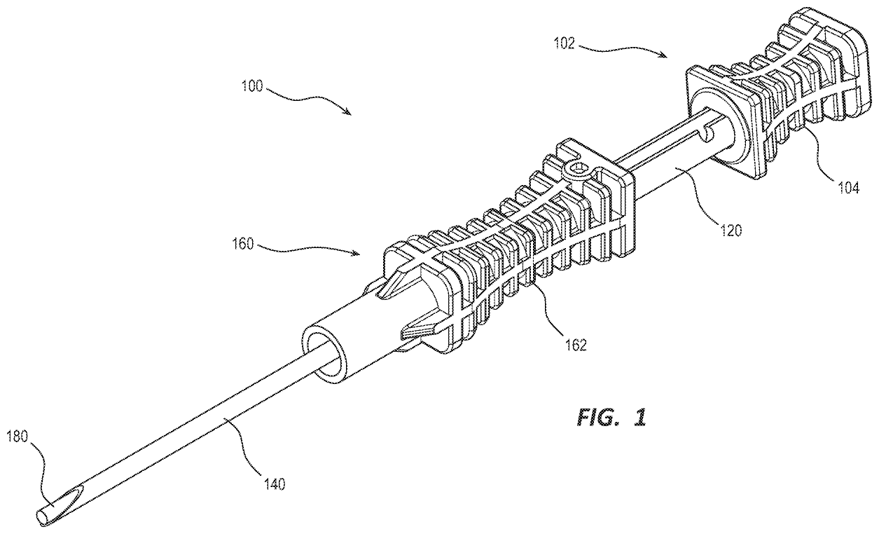

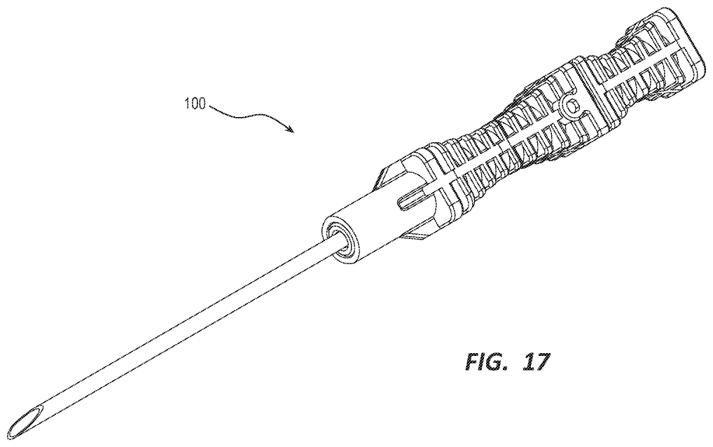

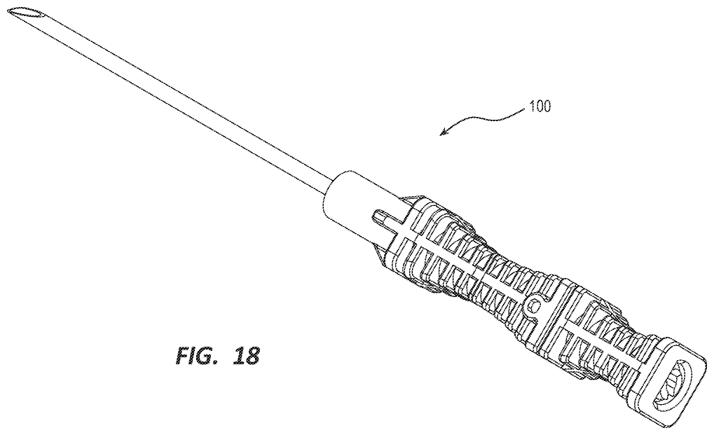

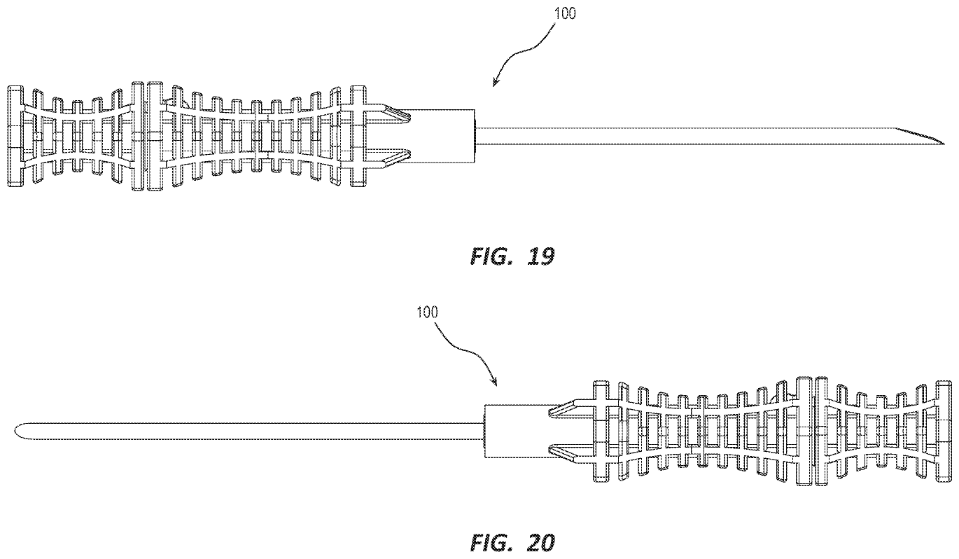

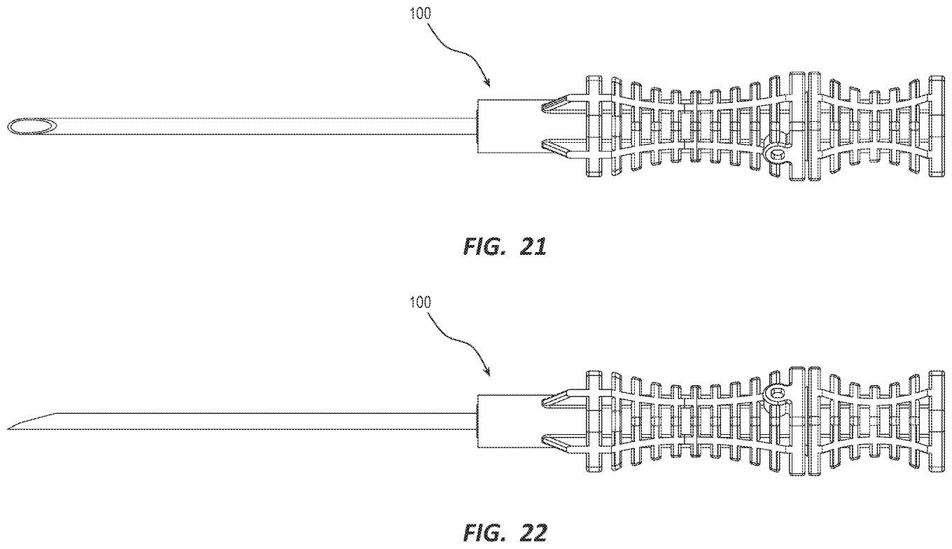

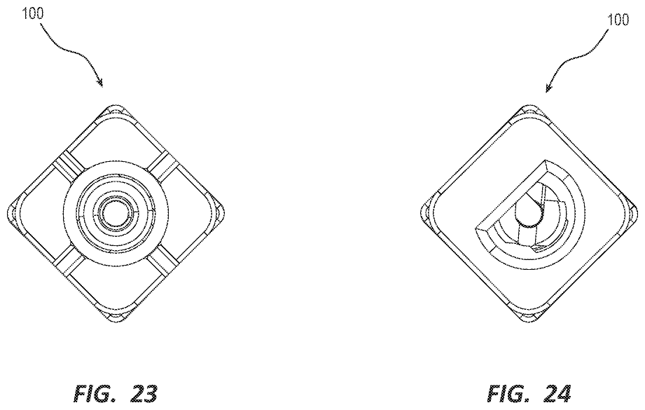

FIG. 17 is a perspective view of the marker insertion device of FIG. 1.

FIG. 19 is a right side view of the marker insertion device of FIG. 1.

FIG. 20 is a back side view of the marker insertion device of FIG. 1.

FIG. 21 is a front view of the marker insertion device of FIG. 1.

FIG. 22 is a left side view of the marker insertion device of FIG. 1.

FIG. 23 is a bottom view of the marker insertion device of FIG. 1.

FIG. 24 is a top view of the marker insertion device of FIG. 1.

### DETAILED DESCRIPTION

Before a biopsy or surgical procedure to remove a lesion within a breast, such as a lumpectomy procedure, the location of the lesion must be identified. For example, mammography or ultrasound imaging may be used to identify and/or confirm the location of the lesion before a procedure. The resulting images may be used by a surgeon during a subsequent procedure to identify the location of the lesion and guide the surgeon, e.g., during dissection to access and/or remove the lesion. Such images may be two dimensional, and therefore provide limited guidance for localization of the lesion.

To facilitate localization in three dimensions, a marker may be placed with an insertion device to provide localization information during a procedure. For example, an insertion device for a marker may be introduced through a breast into a lesion, and in conjunction with mammography or any other standard imaging technique, a marker may be deployed from the insertion device into the lesion. The insertion device may then be withdrawn, and the position of the marker may be confirmed using mammography. During a subsequent surgical procedure, a hand-held probe may be placed over the breast to identify a location overlying the marker. An incision may be made and the probe may be used to guide excision of the marker and lesion.

Markers and placement devices within the scope of this disclosure may be used for locating lesions and placing markers at any location within the body. Specific examples given herein, such as placement of a marker within breast tissue to identify a breast cancer lesion, may be analogized to placement within other areas of the body.

A known problem with conventional insertion devices is that a marker may not always be accurately delivered to the desired site of the lesion. For example, the marker may be prematurely, and/or incompletely, delivered from the inserter. This may be due to a variety of factors, including but not limited to: the mechanism of delivery used by the inserter, indicators given from the inserter to the user of the device about status of complete or incomplete marker deployment, the ergonomic design of the inserter and ease of use provided to the user in deploying the marker, or any combination of these and additional factors.

Accordingly, there is a need for apparatus and methods for accurately and intuitively placing a marker or other tissue structures in advance of and/or during surgical, diagnostic, or other medical procedures. In some embodiments, devices within the scope of this disclosure may be broadly directed to such a system and methods for inserting these structures, including a marker, into the body of a patient.

The components of the embodiments as generally described and illustrated in the figures herein can be arranged and designed in a wide variety of different configurations. Thus, the following more detailed description of various embodiments, as represented in the figures, is not intended to limit the scope of the present disclosure, but is merely representative of various embodiments. While various aspects of the embodiments are presented in drawings, the drawings are not necessarily drawn to scale unless specifically indicated.

The phrase “coupled to” is broad enough to refer to any suitable coupling or other form of interaction between two or more entities, including mechanical, fluidic and thermal interaction. Thus, two components may be coupled to each other even though they are not in direct contact with each other. The phrases “attached to” or “attached directly to” refer to interaction between two or more entities which are in direct contact with each other and/or are separated from each other only by a fastener of any suitable variety (e.g., mounting hardware or an adhesive). The phrase “fluid communication” is used in its ordinary sense, and is broad enough to refer to arrangements in which a fluid (e.g., a gas or a liquid) can flow from one element to another element when the elements are in fluid communication with each other.

The terms “proximal” and “distal” are opposite directional terms. For example, the distal end of a device or component is the end of the component that is furthest from the practitioner during ordinary use. The proximal end refers to the opposite end, or the end nearest the practitioner during ordinary use.

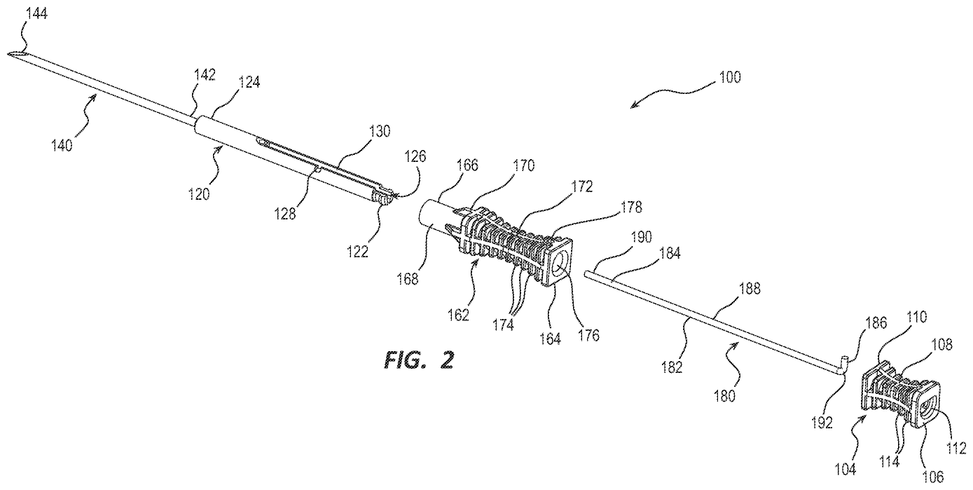

Turning to the drawings, FIGS. 1 and 2 show an exemplary embodiment of a marker insertion device 100, or system for implanting a marker into a patient. Generally, the marker insertion device may include a first assembly 102 comprising a first handle member 104, an elongate member 120, and a cannula 140; and a second assembly 160 comprising a second handle member 162 and a stylet 180. The first handle member 104 may comprise a proximal end 106, a middle portion 108, a distal end 110, a bore 112, and ridges 114. The elongate member 120 may comprise a proximal end 122, a distal end 124, a bore 126, a notch 128, and a channel 130. The channel 130 may comprise a straight portion 132, a raised portion 134, and an end portion 136. The cannula 140 may comprise a proximal end 142, cutting tip 144, bevel 148, void portion 150, and bore 152. The second handle member 162 may comprise a proximal end 164, a distal end 166, a cylindrical portion 168, an intermediate ridge 170, a dip 172, ridges 174, bore 176, and fixing bore 178. The stylet 180 may comprise a longitudinal portion 182 and an engaging portion 184. Engaging portion 184 of stylet 180 may comprise a fixing portion 186, a sliding portion 188, and a distal tip 190. The stylet 180 may further comprise distal tip 190 and bend 192.

These components may be included with or without a marker as described below.

In alternative embodiments, the inserter device and/or inserter device system may or may not comprise a protective cover for a sharp, cutting edge of the insertion device.

In alternative embodiments, the system comprising the components may be included in various types of packaging or transportation and organizational components.

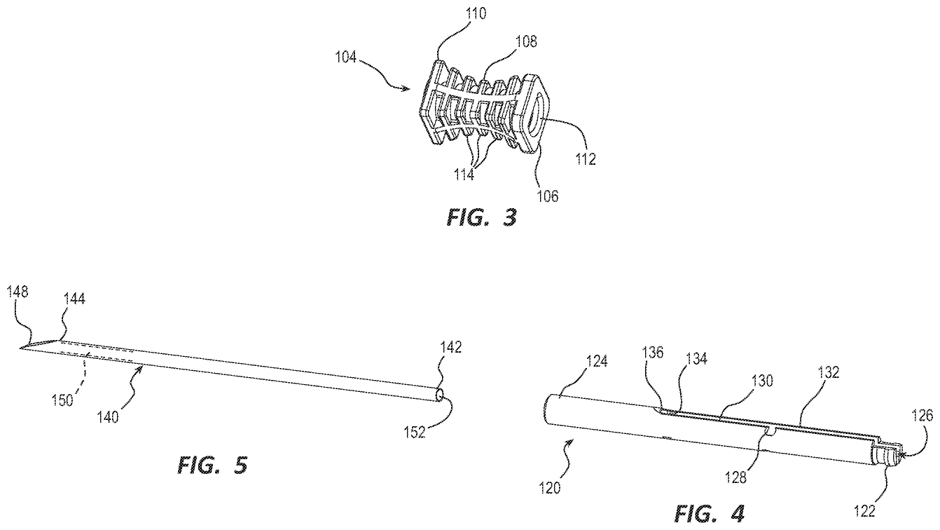

In an exemplary embodiment, FIGS. 2 and 3 illustrate that first handle member 104 may comprise a proximal end 106, a distal end 110, with a bore 112 and an outer middle portion 108 extending between the two. Bore 112 may extend from proximal end 106 to distal end 110, in a longitudinal direction through a longitudinal axis of first handle member 104. Middle portion 108 may further comprise ridges 114 that face radially outward to provide texture or grip to the user of the device. Middle portion 108 may further comprise a dip in profile to further provide a secure grip to the user of the device. It is recognized that a person of ordinary skill in the art, having the benefit of this disclosure, could easily provide multiple profiles for the middle portion 108 of first handle member 104 to provide enhanced grip and a non-slip profile for the user.

In an exemplary embodiment, FIGS. 2 and 4 show elongate member 120. Elongate member 120 may comprise a proximal end 122, a distal end 124, and a bore 126 extending between each end. Bore 126 may comprise a constant diameter and extend from proximal end 122 to distal end 124 through a longitudinal axis of elongate member 120.

Elongate member 120 may further comprise channel 130 that extends a portion of the distance between the proximal end 122 and distal end 124. Channel 130 may begin at proximal end 122 and extend some distance distally in the longitudinal direction. In some embodiments, channel 130 may be a slit in elongate member 120 that extends in the longitudinal direction. In some embodiments, channel 130 may extend from the outer, exterior wall of elongate member 120, to the inner wall of bore 126.

In some embodiments channel 130 may comprise a straight portion 132 that comprises an inner two walls of the channel that extend in the longitudinal direction. The two walls of straight portion 132 may be some distance apart. Channel 130 may also comprise one or more raised portions 134 on straight portions 132 that constrict the channel 130, or lower the distance between the two walls of straight portion 132. Channel 130 may further comprise an end portion 136 that is distal raised portions 134 wherein the two walls of the channel are the same distance apart as straight portion 132.

In some embodiments of the device, elongate member 120 may comprise a notch 128 adjacent the channel 130. Notch 128 may extend from a circumferential, exterior surface of elongate member 120 to the interior bore 126. Notch 128 may be circular, square, or some other shape. In some embodiments, notch 128 may share a portion of its side wall with channel 130. That is to say, a gap in the sidewall of notch 128 may also be an opening in channel 130 such that notch 128 and channel 130 are connected.

In an exemplary embodiment, FIGS. 2 and 5 show cannula 140. Cannula 140 may comprise a proximal end 142 and a distal cutting tip 144. Cutting tip 144 may comprise a sharp bevel 148 that aids in cutting upon insertion of the device into a patient's body. Cannula 140 may further comprise a bore 152 which spans the longitudinal length of the cannula. A distal portion of the bore may be referred to as the void portion 150, and in some embodiments void portion 150 may be configured to hold a marker before insertion into a patient's body.

Cannula 140 may also comprise depth markers to indicate to a user the distance that the cannula 140 has been inserted into a patient's body.

Cannula 140 may also comprise a radiopaque portion to render the cannula 140 visible under X-ray, Fluoroscopy, or any number of standard medical imaging techniques.

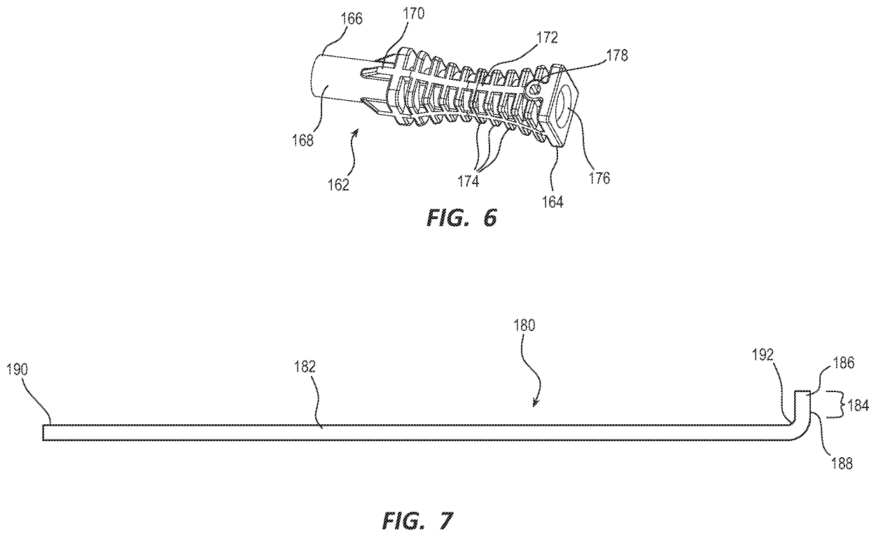

In an exemplary embodiment, seen in FIGS. 2 and 6, second handle member 162 may comprise proximal end 164, distal end 166, and a bore 176 extending longitudinally through second handle member 162. Second handle member 162 may further comprise ridges 174 that span the distance between proximal end 164 and intermediate ridge 170. Ridges 174 may run in the cross-sectional direction and provide a non-slip and enhanced gripping profile for the user. Ridges 174 may also comprise a dip 172 in the profile to provide a non-slip and enhanced gripping profile for the user of the device. It is recognized that a person of ordinary skill in the art, having the benefit of this disclosure, could easily provide multiple profiles for the ridges 174 of second handle member 162 to provide enhanced grip and a non-slip profile for the user.

Second handle member 162 may also comprise a cylindrical portion 168 distal the proximal end 164 and intermediate ridge 170. This cylindrical portion 168 may comprise an external circumference, an inner bore that is a portion of bore 176.

Second handle member 162 may further comprise a fixing bore 178 that may have an axis that is offset from the axis of bore 176. Fixing bore 178 and bore 176 may join at a substantially proximal portion of second handle member 162. The axis of fixing bore 178 and bore 176 may be offset by 180 degrees or less. The diameter of fixing bore 178 may be less than or equal to the diameter of bore 176.

In an exemplary embodiment, FIGS. 2 and 7 show stylet 180 may comprise longitudinal portion 182 and engaging portion 184. Longitudinal portion 182 may have its axis offset from engaging portion 184. Longitudinal portion 182 may have its axis offset from engaging portion through a bend in the stylet of 180 degrees or less. Longitudinal portion 182 may comprise a distal end with a face normal to the longitudinal direction. Stylet 180 may further comprise a circular cross-sectional shape, or a square cross section, or any cross-sectional shape that may be easily extruded.

Engaging portion 184 of stylet 180 may further comprise fixing portion 186 and sliding portion 188. Fixing portion 186 may share a longitudinal axis with sliding portion 188, or the two portions may be axially offset from each other. Fixing portion 186 and sliding portion 188 may be proximal portions of engaging portion 184.

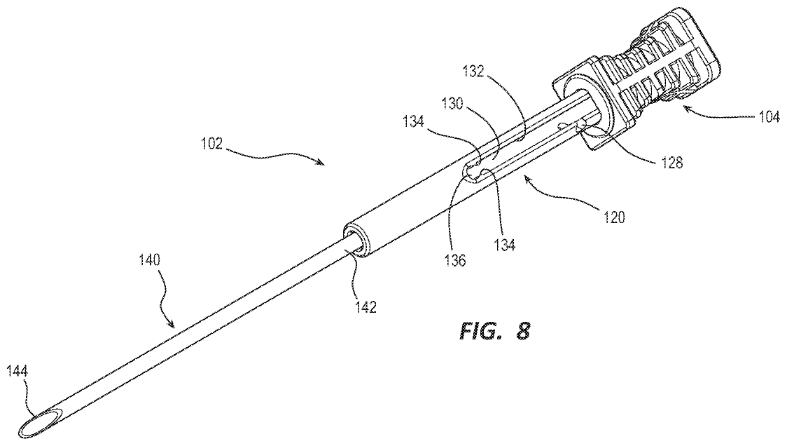

Returning to FIG. 8, when assembled in an exemplary embodiment, first handle member 104 may be coupled to elongate member 120, which is coupled to cannula 140. When connected, these elements can form first assembly 102.

A person of ordinary skill in the art, having the benefit of this disclosure, will recognize that there are multiple methods and mechanisms of coupling first handle member 104 to elongate member 120. However, in at least one embodiment, elongate member 120 may extend from the distal end 110 of first handle member 104. First handle member 104 may be coupled to elongate member 120 by pushing proximal end 122 of elongate member 120 through bore 112 of first handle member 104. Proximal end 122 of elongate member 120 may comprise push tabs to snap into place within bore 112. Alternatively, elongate member 120 and first handle member 104 may be press fit, glued, or comprise any other mechanism of coupling the two portions.

In an exemplary embodiment, elongate member 120 can be coupled to cannula 140. A person of ordinary skill in the art, having the benefit of this disclosure, will recognize that there are multiple methods of coupling elongate member 120 to cannula 140. In at least one embodiment, elongate member 120 may be overmolded onto cannula 140. Other methods of coupling include, but are not limited to, press fitting, gluing, using a tab and slot method, etc.

In one embodiment, due to the couplings above, first handle member 104, elongate member 120, and cannula 140 form one rigid body. This may have the effect that when a user causes first handle member 104 to be moved in the proximal or distal direction, both elongate member 120, and cannula 140 are caused to be moved in the same corresponding direction. Similarly, when a user causes first handle member 104 to be rotated in the clockwise or counterclockwise direction (from the perspective and view of the user, which is in the proximal to distal direction), both elongate member 120, and cannula 140 are caused to be rotated in the same corresponding direction.

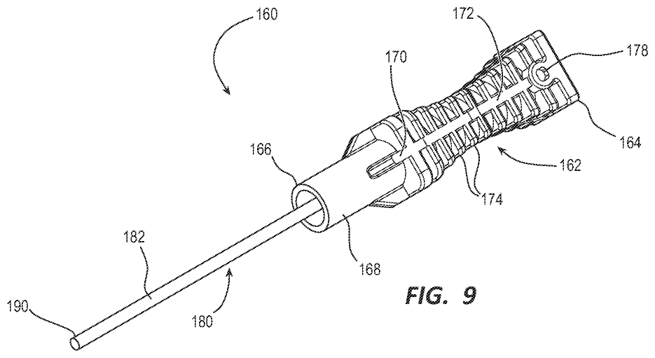

Continuing with FIG. 9, in an exemplary embodiment, second handle member 162 may be coupled to stylet 180. When connected, these elements can form second assembly 160. Multiple methods and mechanisms of coupling second handle member 162 to stylet 180 are within the scope of this disclosure. In an exemplary embodiment, second handle member 162 may be coupled to stylet 180 through press fitting fixing portion 186 of stylet 180 through fixing bore 178 of second handle member 162. This coupling may be such that longitudinal portion 182 of stylet 180 shares a longitudinal axis with second handle member 162. Otherwise stated, longitudinal portion 182 of stylet 180 may be disposed substantially through the center of bore 176 of second handle member 162.

Fixing bore 178 of second handle member 162 may be located in a more proximal portion of second handle member 162. Fixing portion 186 of stylet 180 may be coupled to second handle member 162 at a proximal portion of second handle member 162 such that longitudinal portion 182 of stylet 180 runs through a substantial portion of the length of bore 176 of second handle member 162. The diameter of stylet 180 may be substantially less than bore 176 of second handle member 162 such that there may be a space or void concentrically between the outer circumferential wall of longitudinal portion 182 of stylet 180 and the circumferential walls of bore 176.

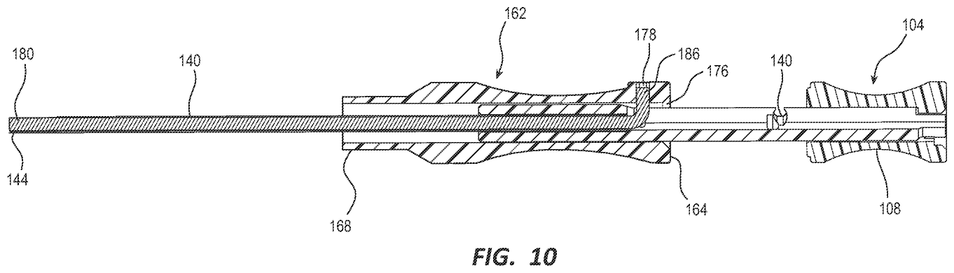

As seen in an exemplary embodiment illustrated in FIG. 10, first assembly 102 and second assembly 160 can be assembled such that an axis is shared, yet the first assembly 102 may be translatable with respect to the second assembly 160 in an axial or longitudinal direction. First assembly 102 and second assembly 160 may also be assembled such that an axis is shared, yet first assembly 102 may be rotatable with respect to the second assembly 160 about their shared axis.

Cannula 140 and elongate member 120 may be disposed or slidingly disposed or rotationally disposed in the space or void concentrically between the outer circumferential wall of longitudinal portion 182 of stylet 180 and the circumferential walls of bore 176. When placed in the assembled configuration, first handle member 104 may proximally abut second handle member 162, and first handle member 104 may be rotatable or translatable with respect to second handle member 162.

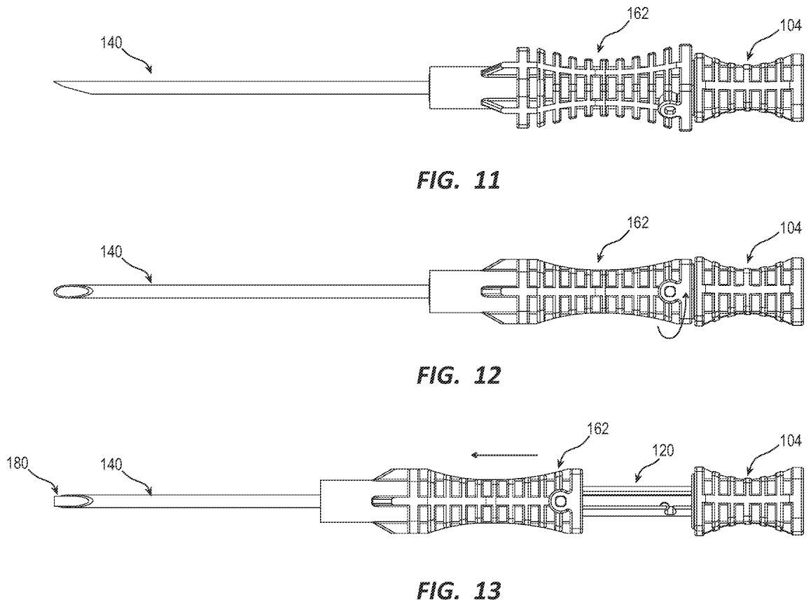

As illustrated in FIGS. 11-13, in an exemplary embodiment, first assembly 102 may rest in a first position, a translating position, or a second position with respect to second assembly 160. Second assembly 160 may be stationary upon operation of the device. First assembly 102 may transition from the first position to an intermediate position, and then to a second position. Each position will be described in further detail below.

As illustrated in the embodiment of the first position seen in FIG. 11, the device may rest in a compacted, or unretracted configuration. First handle member 104 may proximally abut second handle member 162. In this first position, cutting tip 144 of cannula 140 may extend a distance past distal tip 190 of stylet 180. Void portion 150 of the cannula may rest past the distal tip 190 of stylet 180.

In an embodiment of the first position seen in FIG. 11, first handle member 104 may be rotationally offset from second handle member 162. That is to say, ridges 114 and ridges 174 may not align axially. In this position, the device may be configured so as to resist proximal and distal displacement of first handle member 104, and thereby first assembly 102, with respect to second handle member 162 and second assembly 160. However, in this position, first handle member 104, and thereby first assembly 102, may be rotatable with respect to second handle member 162 and second assembly 160, and by this rotation the device can be transitioned out of first position and into a translating position. This rotation may be rotation by 180 degrees or less. Transitioning the device from a first position to a translating position may axially align ridges 114 and ridges 174.

In an embodiment of the translating position seen in FIG. 12, first handle member 104 and thereby first assembly 102, may be translatable in the longitudinal direction with respect to second handle member 162 and thereby second assembly 160. That is to say, first handle member 104 and thereby first assembly 102 may be rotated out of the first position into the translating position, and then retracted proximally with respect to second assembly 160.

As first handle member 104 is retracted proximally, first assembly 102 and thereby elongate member 120 and cannula 140 are also retracted proximally with respect to second assembly 160. Thus, by retraction of first assembly 102 proximally, second assembly 160 takes a respectively more distal position such that distal tip 190 of stylet 180 is displaced distally through cannula 140. This may be such that distal tip 190 passes through void portion 150 of cannula 140 and is driven to protrude past cutting tip 144 in the distal direction, all through retraction of first handle member 104 with respect to second handle member 162.

This displacement of distal tip 190 may serve to eject or push a marker residing in void portion 150 of cannula 140, to a location exterior the cannula 140.

As seen in an embodiment illustrated in FIG. 13, when first handle member 104 has been fully retracted proximally with respect to second handle member 162, the device may be in a second, or fully retracted, position. In the second position, first handle member 104 may not be rotatable with respect to second handle member 162. The most distal portion of cutting tip 144 of cannula 140 may be proximal to distal tip 190 of stylet 180 in this position.

In some embodiments, the marker insertion device 100 may be configured to provide audible and/or tactile feedback as the device is operated and/or displaced between various positions or configurations. For example, audible feedback may be heard by the user when second handle member 162 reaches the most distal second position. Additionally or alternatively, tactile feedback may be felt by the user when second handle member 162 reaches the most distal second position.

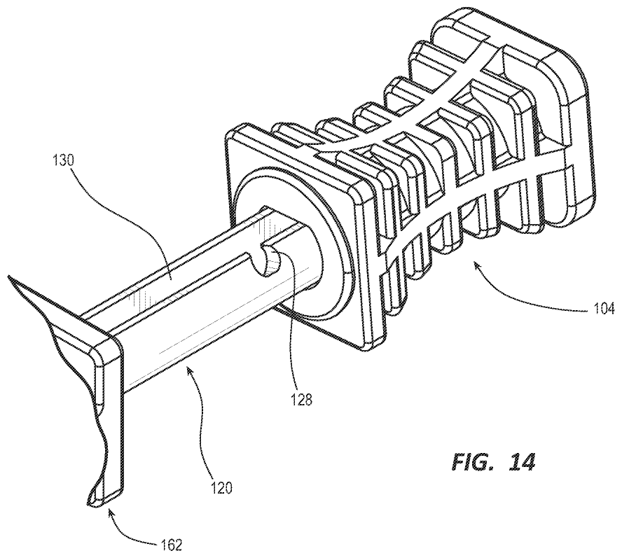

As is seen in FIG. 14, in an embodiment of the device, the device may comprise a locking mechanism to hold the device, including first assembly 102 and second assembly 160, in the first position. In some embodiments this locking mechanism may comprise a notch 128 on elongate member 120 configured to engage with sliding portion 188 of engaging portion 184 of stylet 180. In some embodiments, first assembly 102 may be locked against longitudinal, translational movement through sliding portion 188 of stylet 180 being located in notch 128. By rotating first handle member 104, and thereby rotating first assembly 102, sliding portion 188 of engaging portion 184 is displaced from and disengaged by notch 128, and placed into straight portion 132 of channel 130. In this way, first assembly 102 can be transitioned from a first position to a translating position.

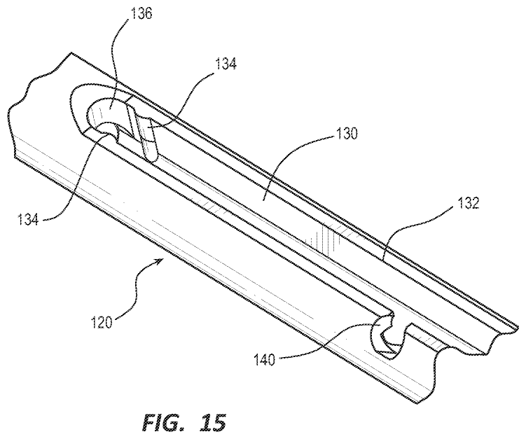

As seen in FIG. 15, in an embodiment of the device, raised portion 134 may provide either, or both, tactile and audible feedback to the user of the device that the device and first assembly 102 has entered the fully retracted, second position. Raised portion 134 may comprise either one, or two, or more elevated portions on the inside wall of channel 130 that frictionally engage with sliding portion 188 of engaging portion 184 of stylet 180. As first assembly 102 is retracted with respect to second assembly 160, raised portion 134 of channel 130 frictionally engage with sliding portion 188 of engaging portion 184 of stylet 180. This frictional engagement can provide either, or both, tactile and audible feedback to the user of the device.

The frictional engagement provided by raised portion 134 may further serve to hold sliding portion 188 at end portion 136 of channel 130 after the device has transitioned to the second position.

Various types of markers can be used in conjunction with the device. A person of ordinary skill in the art will be able to design or envision various other markers and types of markers that take advantage of radar, x-ray, mammography, RFID, or any other imaging and localization techniques typically used in short-distance localization, particularly in the medical field.

Prior to insertion of the device, a marker may be inserted into void portion 150 of cannula 140. This marker may be held in position through friction with the inner walls of bore 152 of cannula 140. A person of ordinary skill in the art will be able to design or envision various other ways of holding the marker in this position within the cannula 140.

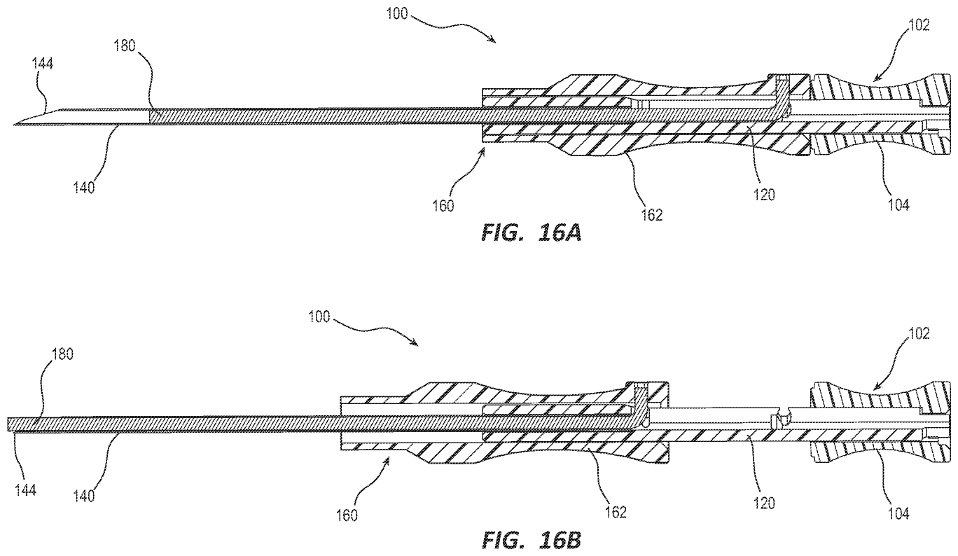

As illustrated in FIGS. 16A and 16B, in an embodiment of the device, when the device is in the fully retracted, or second position, distal tip 190 of stylet 180 extends past the distal most tip of cutting tip 144 of cannula 140 such that the overall end of the device is non-sharp. This serves the purpose of covering the sharp distal tip of the device after the device has been fully retracted, the marker has been inserted, and the device is ready to be removed from the body of a patient.

FIGS. 17-24 are various views of the marker device 100 of FIG. 1. FIG. 17 is a front perspective view of the marker insertion device of FIG. 1. FIG. 18 is a rear perspective view of the marker insertion device of FIG. 1. FIG. 19 is a right side view of the marker insertion device of FIG. 1. FIG. 20 is a back side view of the marker insertion device of FIG. 1. FIG. 21 is a front view of the marker insertion device of FIG. 1. FIG. 22 is a left side view of the marker insertion device of FIG. 1. FIG. 23 is a bottom view of the marker insertion device of FIG. 1. FIG. 24 is a top view of the marker insertion device of FIG. 1.

In an embodiment of the device, the position of the marker within the body of a patient remains the same with respect to the body of the patient while the device is in the first position, transitioning position, and second position. That is to say, the marker remains immobile, as cannula 140 is retracted from around the marker. In this way, the marker remains immobile through deployment from the device, and the following retraction of the device. This lack of movement provides added precision to the user in placing the marker at a specific location in the patient's body.

In some embodiments of the device, a user may insert the device 100 of FIG. 1 into the body of a patient while the device is in the first position. The user may employ any type of standard imaging technique to help guide the user to the correct location and where the marker is desired to be placed. Once the user has placed the distal tip of cutting tip 144 of cannula 140 at the desired location, the user may then rotate first handle member 104 to transition the device into the translating position. The user may then retract first handle member 104 proximally and first assembly 102 until the first handle member 104 and first assembly 102 are in the second position. This may deploy the marker from the device and leave the marker embedded at the target site. The user may then retract the entire device from the body of the patient.

Any methods disclosed herein include one or more steps or actions for performing the described method. The method steps and/or actions may be interchanged with one another. In other words, unless a specific order of steps or actions is required for proper operation of the embodiment, the order and/or use of specific steps and/or actions may be modified. Moreover, sub-routines or only a portion of a method described herein may be a separate method within the scope of this disclosure. Stated otherwise, some methods may include only a portion of the steps described in a more detailed method.

Reference throughout this specification to “an embodiment” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated by one of skill in the art with the benefit of this disclosure that in the above description of embodiments, various features are sometimes grouped together in a single embodiment, figure, or description thereof for the purpose of streamlining the disclosure. This method of disclosure, however, is not to be interpreted as reflecting an intention that any claim requires more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

Recitation in the claims of the term “first” with respect to a feature or element does not necessarily imply the existence of a second or additional such feature or element. It will be apparent to those having skill in the art that changes may be made to the details of the above-described embodiments without departing from the underlying principles of the present disclosure.

We claim:

1. A system for implanting a marker, the system comprising:
a marker insertion device comprising:
a first handle member coupled to a cannula,
a second handle member coupled to a stylet, the stylet disposed within the cannula, and
an elongate member configured to retain the first handle member in a first position relative to the second handle member.

2. The system of claim 1, wherein the elongate member is further configured to indicate to a user when the first handle member is moved from a first position to a second position relative to the second handle member.

3. The system of claim 2, wherein the elongate member is configured to provide a tactile indication when the first handle member is moved into the second position.

4. The system of claim 2, wherein the elongate member is configured to provide an audible indication when the first handle member is moved into the second position.

5. The system of claim 1, wherein the stylet comprises a longitudinal portion and an engaging portion.

6. The system of claim 5, wherein the stylet is coupled to the second handle member at the engaging portion.

7. The system of claim 5, wherein the elongate member further comprises a channel configured to slidingly engage with the engaging portion of the stylet.

8. The system of claim 5, wherein the elongate member further comprises a notch configured to rotatingly engage with engaging portion of the stylet.

9. The system of claim 5, wherein the engaging portion of the stylet comprises a bend of approximately 90 degrees to the longitudinal portion or the stylet.

10. The system of claim 5, wherein the elongate member further comprises a notch that engages with the engaging portion of the stylet such that the elongate member is immovable in the longitudinal direction with respect to the stylet.

11. The system of claim 10, wherein the elongate member is configured to rotate about its longitudinal axis such that the engaging portion of the stylet is displaced from the notch into the channel.

12. The system of claim 1, wherein the elongate member is configured to be rotatable from the first position, wherein the elongate member is immovable in the longitudinal direction, to an intermediate position, wherein the elongate member is movable in the longitudinal direction.

13. The system of claim 1, wherein the distal tip of the cannula comprises a void configured to receive a marker.

14. The system of claim 1, wherein the system further comprises a marker.

15. A system for implanting a marker, the system comprising:
a marker insertion device comprising:
a first handle member coupled to a cannula,
a second handle member coupled to a stylet within the cannula,
an elongate member configured to indicate to a user when the first handle member is moved from a first position to a second position relative to the second handle member.

16. The system of claim 15, wherein the elongate member is further configured to retain the first handle member in the first position relative to the second handle member.

17. The system of claim 15, wherein in the second position of the first handle member the first handle member is retracted to be a distance proximally from the second handle member.

18. The system of claim 15, wherein the elongate member is configured to be rotatable from the first position, wherein the elongate member is immovable in the longitudinal direction, to an intermediate position, wherein the elongate member is movable in the longitudinal direction.

19. The system of claim 18, wherein the channel is configured to frictionally engage with the stylet.

20. A method for implanting a marker within a patient's body, comprising:
obtaining a marker
obtaining a marker insertion device comprising:
a first handle member coupled to a cannula,
a second handle member coupled to a stylet within the cannula,
an elongate member configured to hold the first handle member in a first position relative to the second handle member, and further configured to indicate to a user when the first handle member is in a second position relative to the second handle member;
inserting the insertion device into the patient's body while the first handle member is in the first position,
rotating the first handle member and thereby the elongate member such that the handle member is rotated from a first position to an intermediate position,
displacing the first handle member proximally until the elongate member indicates that the first handle member is in the second position,
removing the marker insertion device from the patient's body.
